(19) United States
(12) Patent Application Publication
(10) Pub. No.: US 2024/0407981 A1
(43) Pub. Date: Dec. 12, 2024

(54) Crushable Capsule Beads Containing Cuticle Moisturizing Oils

(71) Applicants: Donnie Shih-Chieh Chen, Ajax (CA); Danio Gar Ming Kwan, Ajax (CA)

(72) Inventors: Donnie Shih-Chieh Chen, Ajax (CA); Danio Gar Ming Kwan, Ajax (CA)

(21) Appl. No.: 18/208,279

(22) Filed: Jun. 11, 2023

(51) Int. Cl. A61K 8/11; A61K 8/65; A61K 8/73; A61Q 19/00

(52) U.S. Cl. CPC A61K 8/11; A61K 8/65; A61K 8/731; A61Q 19/007; A61K 2800/522; A61K 2800/652; A61K 2800/654

(57) ABSTRACT

Embodiments of the present disclosure encompass crushable capsule beads containing cuticle moisturizing oils, comprising a biodegradable polymer shell, and a core comprising a blend of cuticle moisturizing oils and additional stabilizers or additives. The biodegradable polymer shell may be made from gelatin, cellulose, agar, gellan gum, carrageenan, or other suitable materials. The cuticle moisturizing oils may include jojoba oil, almond oil, vitamin E oil, and avocado oil. The crushable capsule beads provide protection against oxidation, enhanced freshness, and portability, making them a reliable and convenient option for on-the-go cuticle care.

### BACKGROUND OF THE INVENTION

### Field of the Invention

The present invention pertains to the field of cosmetics and introduces a novel composition of crushable capsule beads that contain oils for moisturizing cuticles. These capsules offer unique advantages, such as protection against oxidation and maintaining the freshness of the oils. The disclosed invention encompasses crushable capsule beads containing cuticle moisturizing oils, including a biodegradable polymer shell, as well as a core comprising a blend of cuticle moisturizing oils and additional stabilizers or additives. The invention also covers other embodiments described within the application, including the crushable capsule beads according to claim 1.

### Description of Related Art

Maintaining well-hydrated and healthy cuticles plays a vital role in the overall appearance and wellness of nails. Various cosmetic products have been developed to address this need, such as creams, lotions, and oils. However, existing formulations often suffer from limitations in terms of convenience, portability, or efficacy. Furthermore, the exposure of oils to oxygen can lead to rancidification, diminishing their effectiveness and potentially causing harm.

### SUMMARY OF THE INVENTION

The present invention introduces a novel composition in the form of crushable capsule beads that contain oils specially formulated for cuticle moisturization. These beads are designed to release the oils upon crushing, providing a convenient and mess-free application method. Moreover, the outer shell of the capsule acts as a protective barrier, preventing oxidation and ensuring the freshness and efficacy of the oils.

In certain embodiments, the biodegradable polymer shell may be constructed using materials such as gelatin, cellulose, agar, gellan gum, carrageenan, or other suitable substances. The invention also covers the crushable capsule beads according to claim 1. Additionally, the disclosed invention includes but is not limited to various cuticle moisturizing oils, such as jojoba oil, almond oil, vitamin E oil, and avocado oil.

The invention offers several advantages, including protection against oxidation, enhanced freshness, portability, and convenience. The capsule's outer shell provides protection, preventing oxidation of the contained oils and maintaining their quality and efficacy. By minimizing oxidation, the invention ensures the freshness and effectiveness of each application. The crushable capsule beads also maintain the oils in a controlled environment, away from oxygen exposure, preventing premature degradation and ensuring consistent and effective cuticle moisturization. Furthermore, the compact and travel-friendly nature of the crushable capsule beads allows users to carry them conveniently in purses, pockets, or cosmetic bags. The mess-free application and protection against oxidation make them a convenient and reliable option for on-the-go cuticle care.

### DESCRIPTION OF THE DRAWINGS

FIG. 1 is a block diagram illustrating crushable capsule beads, according to some embodiments of the present disclosure. The crushable capsule beads (110) comprise cuticle moisturizing oils (111), a biodegradable polymer shell (112), a core (113) containing a blend of cuticle moisturizing oils and additional stabilizers or additives, protection against oxidation (115), enhanced freshness (116), portability and convenience (117), and other components as described in claim 1.

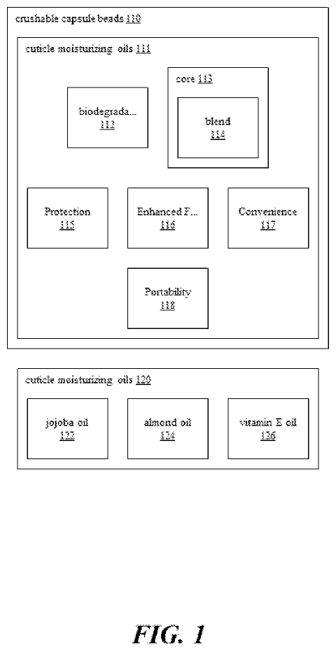

### DETAILED DESCRIPTION OF THE INVENTION

The present invention provides a unique solution for moisturizing cuticles through the utilization of crushable capsule beads. These beads are composed of a combination of cuticle moisturizing oils and other beneficial ingredients to enhance the overall efficacy of the product. The manufacturing process and specific components of the invention are described below.

### Composition of Crushable Capsule Beads

The crushable capsule beads consist of the following elements:
1.1. Shell Material: The outer shell of the capsule beads is made from a biodegradable polymer, such as gelatin, cellulose, agar, gellan gum, carrageenan, or other suitable materials. This capsule skin acts as a protective barrier against oxidation, ensuring the freshness and effectiveness of the contained oils.
1.2. Cuticle Moisturizing Oils: The core of the capsule beads contains a blend of cuticle moisturizing oils, including but not limited to jojoba oil, almond oil, vitamin E oil, and avocado oil. These oils are known for their hydrating and nourishing properties, providing essential nutrients to the cuticles.
1.3. Stabilizers and Additives: The capsule bead composition may also include stabilizers, preservatives, emulsifiers, and other additives commonly used in cosmetic formulations. These ingredients ensure product stability, shelf-life, and ease of application.

### Manufacturing Process

The manufacturing process for the crushable capsule beads involves the following steps:
2.1. Preparation of Shell Material: The biodegradable polymer is melted and formed into small spherical shapes using encapsulation equipment or similar techniques. This controlled manufacturing process ensures uniformity in size and thickness, providing consistent protection against oxidation.
2.2. Oil Filling: The cuticle moisturizing oils, along with any additional stabilizers or additives, are carefully injected into the preformed shells using suitable encapsulation machinery. The oil volume in each bead is controlled to ensure a consistent dosage upon crushing.
2.3. Capsule Sealing: Once the oil filling is complete, the open ends of the capsule shells are sealed, effectively encapsulating the oils and providing a protective barrier against oxidation.

### Usage and Application

To use the crushable capsule beads, the user simply takes one or more beads between their fingertips and applies gentle pressure to crush the beads. This releases the encapsulated oils, which can then be directly applied to the cuticles. The crushed beads disintegrate upon pressure, leaving behind the moisturizing oils without any residue or mess.

### Advantages of the Invention

The present invention offers several advantages over existing cuticle moisturizing products:
4.1. Protection Against Oxidation: The capsule's outer shell acts as a protective barrier, preventing the oils and other liquids contained in the capsule from oxidation. This safeguards the oils against rancidification, which occurs when fats in the oil break down and produce harmful free radicals. By minimizing oxidation, the invention ensures the freshness and efficacy of every application.
4.2. Enhanced Freshness: The crushable capsule beads maintain the oils in a controlled environment, away from oxygen exposure. This prevents premature degradation and maintains the quality and potency of the oils, allowing for consistent and effective cuticle moisturization.
4.3. Portability and Convenience: The crushable capsule beads are compact and travel-friendly, allowing users to carry them conveniently in purses, pockets, or cosmetic bags. The mess-free application and protection against oxidation make them a convenient and reliable option for on-the-go cuticle care.

### CONCLUSION

The described invention presents a novel composition of crushable capsule beads containing cuticle moisturizing oils, offering improved convenience, efficacy, and protection against oxidation for users. This invention opens up new possibilities in the field of cosmetics and contributes to the overall well-being of individuals by ensuring the freshness and effectiveness of cuticle moisturization.

I hereby claim:

1. Crushable capsule beads containing cuticle moisturizing oils, comprising:
a. A biodegradable polymer shell;
b. A core comprising a blend of cuticle moisturizing oils and additional stabilizers or additives.

2. The crushable capsule beads according to claim 1, wherein the biodegradable polymer shell is made from gelatin, cellulose, agar, gellan gum, carrageenan, or other suitable materials.

3. The crushable capsule beads according to claim 1, wherein the cuticle moisturizing oils comprise jojoba oil, almond oil, vitamin E oil, and avocado oil.
